(19) United States
(12) Patent Application Publication
(10) Pub. No.: US 2020/0129396 A1
(43) Pub. Date: Apr. 30, 2020

(54) STYLING PRODUCT FOR HEAD HAIR AND/OR BEARD HAIR II

(71) Applicant: Henkel AG & Co. KGaA, Duesseldorf (DE)

(72) Inventors: NORA KOOPMANN, Hamburg (DE); SANDRA BRANDT, Pinneberg (DE); MARCUS NOLL, Quickborn (DE)

(73) Assignee: Henkel AG & Co. KGaA, Duesseldorf (DE)

(21) Appl. No.: 16/654,976

(22) Filed: Oct. 16, 2019

(30) Foreign Application Priority Data
Oct. 29, 2018 (DE) 10 2018 218 511.5

(51) Int. Cl. A61K 8/34; A61K 8/04; A61K 8/92; A61K 8/362; A61Q 5/06

(52) U.S. Cl. CPC A61K 8/342; A61K 8/345; A61Q 5/06; A61K 8/92; A61K 8/362; A61K 8/04

(57) ABSTRACT

The application describes a cosmetic composition for temporarily deforming hair, comprising—in each case in relation to the total weight of the composition—a) from about 20 to about 60% by weight water, b) from about 2 to about 20% by weight of at least one polyvalent alcohol, and c) from about 20 to about 65% by weight of at least one alkoxylated fatty alcohol.

### CROSS-REFERENCE TO RELATED APPLICATION

This application claims priority to German Patent Application No. 10 2018 218 511.5, filed Oct. 29, 2018, which is incorporated herein by reference in its entirety.

### TECHNICAL FIELD

The present application describes a cosmetic composition for temporarily deforming hair, which cosmetic composition comprises water, a polyvalent alcohol and an alkoxylated fatty alcohol, and also to the use of said cosmetic composition. In addition, a method for temporarily deforming hair with use of the cosmetic composition is disclosed.

### BACKGROUND

With the comeback of a full beard and barber shops, pomade has also been enjoying renewed popularity in the field of male grooming for some time.

Pomade has existed since the 18^{th} century and was obtained by way of a complex process from apples (“pomo” in Italian and “pomme” in French).

Pomades were developed primarily to make the hair look smooth and at the same time to provide a shiny, but not oily finish. Pomades are suitable in principle in particular for hairstyles that are formed using a comb. In contrast to gels, pomades do not dry out or leave behind a hard film on the hair. The hair can thus be styled over and over again throughout the day.

Pomades are usually free from alcohol and synthetic styling polymers (=film-forming polymers) and therefore do not dry out the hair. Pomade is usually an ointment-like substance including various fats and exists in oil-based form and in water-based form. The latter can be removed in a more versatile manner and more easily. Oil-based products hold the hairstyle better, but are more difficult to remove from the hair.

There is still a need for styling products for head hair and/or beard hair that bestow a high holding power on the hair treated with them and at the same time do not contain any synthetic, film-forming polymers. In addition, these styling products should bestow a natural, non-greasy or oily shine on the hair treated with them.

### BRIEF SUMMARY

This object is achieved by a cosmetic composition for temporarily deforming hair, comprising—in each case in relation to the total weight of the composition—

a) from about 20 to about 60% by weight water,
b) from about 2 to about 20% by weight of at least one poly valent alcohol, and
c) from about 20 to about 65% by weight of at least one alkoxylated fatty alcohol.

It has surprisingly been found that such a cosmetic composition for temporarily deforming hair, in spite of the absence of synthetic, film-forming polymers, bestows a high holding power on hair treated with it, without leaving the hair looking oily or greasy. In addition, such a composition can be distributed in the hair very well and uniformly.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The cosmetic composition for temporarily deforming hair contains water as first essential ingredient. The amount of water is at least about 20% by weight, in relation to the total weight of the composition. Accordingly, the composition is a water-based cosmetic composition. The amount of water in the cosmetic composition is preferably at least about 30% by weight, and even more preferably at least about 35% by weight, in each case in relation to the total weight of the cosmetic composition. Particularly preferred cosmetic compositions are exemplified accordingly in that the amount of water is from about 30 to about 60% by weight water and more preferably from about 35 to about 60% by weight, in each case in relation to the total weight of the cosmetic composition.

The cosmetic composition contains at least one polyvalent alcohol as second essential ingredient.

The polyvalent alcohol is preferably selected from the group including 1,2-propanediol, 1,3-propanediol, 2-methyl-1,3-propanediol, glycerol, 1,2-butylene glycol, 1,3-butylene glycol, 1,4-butylene glycol, 1,2-pentanediol, 1,5-pentanediol, 1,2-hexanediol, 1,6-hexanediol, 2-methyl-2,4-pentanediol, 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomer mixtures of eis- and trans-1,4-dimethylolcyclohexane, PEG-3, PEG-4, PEG-6, PEG-7, PEG-8, PEG-9, PEG-10, PEG-12, PEG-14, PEG-16, PEG-18 and PEG-20 and mixtures thereof.

Particularly preferred polyvalent alcohols are selected from 1,2-propanediol, glycerol, 1,3-butylene glycol, 1,6-hexanediol, and sorbitol and mixtures hereof. Glycerol or a mixture of glycerol and 1,2-propanediol is exceptionally preferred.

The amount of the at least one polyvalent alcohol is preferably from about 4 to about 18% by weight and more preferably from about 6 to about 15% by weight, in each case in relation to the total amount of cosmetic agent.

Preferred cosmetic compositions contain from about 6 to about 15% by weight glycerol as polyvalent alcohol.

Cosmetic compositions that likewise are preferred contain from about 6 to about 15% by weight glycerol and from about 0.5 to about 3% by weight 1,2-propanediol as poly valent alcohol.

The cosmetic composition contains at least one alkoxylated fatty alcohol as third essential ingredient.

An “alkoxylated fatty alcohol” is any fatty alcohol with a carbon chain of C5 or more, which also comprises at least one alkoxy group. The fatty alcohol can be selected for example from C₉-C₁₁ fatty alcohols, C₁₂-C₁₃ fatty alcohols, C₁₂-C₁₅ fatty alcohols, C₁₂-C₁₆ fatty alcohols, C14-C5 fatty alcohols, arachidyl alcohol, behenyl alcohol, capryl alcohol, cetearyl alcohol, cetyl alcohol, coconut alcohol, decyl alcohol, (hydrated) tallow alcohol, lauryl alcohol, myristyl alcohol, oleyl alcohol, palm alcohol, palm kern alcohol, stearyl alcohol and tridecyl alcohol.

The alkoxy group can comprise in particular ethoxy and/or propoxy and/or butoxy groups. The alkoxylated fatty alcohol is particularly preferably an ethoxylated fatty alcohol.

The at least one alkoxy group of the “alkoxylated fatty alcohol” can be derived for example from an alkoxylation reaction with alkylene oxide, in particular ethylene oxide and/or propylene oxide.

It is preferred that the at least one alkoxylated fatty alcohol is selected from the group including Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Laureth-1, Laureth-2, Laureth-3, Laureth-4, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-23, Laureth-25, Laureth-30, Laureth-40, Deceth-3, Deceth-5, Oleth-5, Oleth-30, Steareth-2, Steareth-4, Steareth-6, Steareth-7, Steareth-10, Steareth-11, Steareth-13, Steareth-14, Steareth-15, Steareth-20, Steareth-21, Steareth-25, Steareth-27, Steareth-30, Steareth-40, Steareth-50, Steareth-100 and mixtures thereof.

The name Ceteareth-2 for example stands for a C16-Cis fatty alcohol with an average of 2 ethylene oxide units per molecule.

In a very particularly preferred embodiment of the cosmetic composition the at least one alkoxylated fatty alcohol comprises Ceteareth-25. In a very particularly preferred embodiment of the cosmetic composition the at least one alkoxylated fatty alcohol is Ceteareth-25.

The amount of the at least one alkoxylated fatty alcohol is preferably from about 20 to about 50% by weight and more preferably from about 25 to about 40% by weight, in each case in relation to the total amount of cosmetic composition.

In an exceptionally preferred embodiment of the cosmetic composition it contains from about 20 to about 50% by weight and more preferably from about 25 to about 40% by weight of Ceteareth-25 as at least one alkoxylated fatty alcohol, in each case in relation to the total amount of cosmetic composition.

The cosmetic composition is preferably free from synthetic, film-forming polymers. The expression “free from” means that the cosmetic composition contains at most about 0.2% by weight and preferably 0% by weight of synthetic, film-forming polymers, in each case in relation to the total weight of the composition.

In a preferred embodiment the composition also comprises at least one oil.

Within the scope of this application an oil refers to a compound that is not miscible or is only miscible to a limited extent with water and that is liquid at about 25° C.

Suitable oils are selected from the esters of linear or branched saturated or unsaturated fatty alcohols with 2-30 carbon atoms with linear or branched saturated or unsaturated fatty acids with 2-30 carbon atoms, which may be hydroxylated. These include cetyl-2-ethylhexanoate, 2-hexyldecylstearate (for example Eutanol® G 16 S), 2-hexyldecyllaurate, isodecylneopentanoate, isononylisononanoate, 2-ethylhexylpalmitate (for example Cegesoft® C 24) and 2-ethylhexylstearate (for example Cetiol® 868). Likewise preferred are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate.

Further preferred oils are selected from natural and synthetic hydrocarbons, particularly preferably from mineral oils, paraffin oils, C₁₈-C₃₀ isoparaffins, in particular isoeicosane, polyisobutenes and polydecenes, which for example are obtainable under the name Emery® 3004, 3006, 3010 or under the name Ethylflo® from Albemarle or Nexbase® 2004G from Nestle, furthermore selected from C5-C16 isoparaffins, in particular from isodecane, isododecane, isotetradecane and isohexadecane and mixtures hereof, and 1,3-di-(2-ethylhexyl)-cyclohexane (obtainable for example under the trade name Cetiol® S from BASF).

Further preferred oils are selected from the benzoic acid esters of linear or branched C8-22 alkanols. Particularly preferred are benzoic acid C12-C15 alkyl esters, obtainable for example as the trade product Finsolv® TN, benzoic acid isostearyl esters, obtainable for example as the trade product Finsolv® SB, ethyl hexyl benzoate, obtainable for example as the trade product Finsolv® EB, and benzoic acid octyl docecyl esters, obtainable for example as the trade product Finsolv® BOD.

Further preferred oils are selected from fatty alcohols with 6-30 carbon atoms which are unsaturated or branched and saturated or branched and unsaturated. The branched alcohols are often also referred to as Guerbet alcohols since they are obtainable by the Guerbet reaction. Preferred alcohol oils are 2-hexyl decanol (Eutanol® G 16), 2-octyl dodecanol (Eutanol® G), 2-ethylhexyl alcohol and isostearyl alcohol.

Further preferred oils are selected from mixtures of Guerbet alcohols and Guerbet alcohol esters, for example the trade product Cetiol® PGL (2-hexyldecanol and 2-hexyldecyl laurate).

Further preferred oils are selected from the triglycerides (=triple esters of glycerol) or linear or branched, saturated or unsaturated, optionally hydroxylated C8-30 fatty acids. The use of natural oils, for example amaranth seed oil, apricot kernel oil, argan oil, avocado oil, babassu oil, cottonseed oil, borage seed oil, camelina oil, safflower oil, peanut oil, pomegranate seed oil, grapefruit seed oil, hemp oil, hazelnut oil, elderberry seed oil, currant seed oil, jojoba oil, cocoa butter, linseed oil, macadamia nut oil, corn oil, almond oil, marula oil, evening primrose oil, olive oil, palm oil, palm kernel oil, Brazil nut oil, pecan nut oil, peach stone oil, rapeseed oil, rice bran oil, sea buckthom berry oil, sea buckthom seed oil, sesame oil, soybean oil, sunflower oil, grape seed oil, walnut oil, wild rose oil, wheatgerm oil and the like. Synthetic triglyceride oils, however, in particular capric/caprylic triglycerides, for example the trade products Myritol® 318, Myritol® 331 (BASF) or Miglyol® 812 (Huls) with unbranched fatty acid esters and glyceryl triisostearin with branched fatty acid esters are preferred.

Further preferred oils are selected from the dicarboxylic acid esters of linear or branched C2-C10 alkanols, in particular diisopropyl adipate, di-n-butyl adipate, di-(2-ethylhexyl)adipate, dioctyl adipate, diethyl-/di-n-butyll dioctyl sebacate, diisopropyl sebacate, dioctyl malate, dioctyl maleate, dicaprylyl maleate, diisooctyl succinate, di-2-ethylhexyl succinate and di-(2-hexyldecyl)-succinate.

Further preferred oils are selected from the addition products of from about 1 to about 5 propylene oxide units of mono- or polyvalent C5-C22 alkanols, such as octanol, decanol, decanediol, lauryl alcohol, myristyl alcohol and stearyl alcohol, for example PPG-2-myristyl ether and PPG-3-myristyl ether (Witconol® APM).

Further preferred oils are selected from the addition products of at least about 6 ethylene oxide and/or propylene oxide units of mono- or polyvalent C3-C22 alkanols such as glycerol, butanol, butanediol, myristyl alcohol and stearyl alcohol, which may be esterified as desired, for example PPG-14 butyl ether (Ucon Fluid® AP), PPG-9 butyl ether (Breox® B25), PPG-10 butanediol (Macol® 57), PPG-15 stearyl ether (Arlamol® E) and glycereth-7-diisononanoate.

Further preferred oils are selected from the C5-C22 fatty alcohol esters of monovalent or polyvalent C2-C7 hydroxycarboxylic acids, in particular the esters of glycolic acid, lactic acid, malic acid, tartaric acid, citric acid and salicylic acid. Such esters based on linear C14/15 alkanols, for example C12-C15 alkyl lactate, and C12/13 alkanols branched in the 2-position, are obtainable under the trade name Cosmacol® from Nordmann, Rassmann GmbH & Co, Hamburg, in particular the trade products Cosmacol® ESI, Cosmacol® EMI and Cosmacol® ETI.

Further preferred oils are selected from the symmetrical, asymmetrical or cyclic esters of carbonic acid with C3-22 alkanols, C3-22 alkanediols or C3-22 alkanetriols, for example dicaprylyl carbonate (Cetiol® CC) or glycerol carbonate.

Further oils that may be preferred are selected from the esters of dimers of unsaturated C12-C22 fatty acids (dimer fatty acids) with monovalent linear, branched or cyclic C2-Cis alkanols or with polyvalent linear or branched C2-C6 alkanols.

Further oils that are suitable are selected from the silicone oils, which for example include dialkyl- and alkylaryl siloxanes, such as cyclopentasiloxane, cyclohexasiloxane, dimethylpolysiloxane and methylphenylpolysiloxane, but also hexamethyldisiloxane, octamethyltrisiloxane and decamethyltetrasiloxane. Volatile silicone oils which may be cyclic may be preferred, for example octamethylcyclotetrasiloxane, decamethylcyclopentasiloxane and dodecamethylcyclohexasiloxane and mixtures hereof, as are contained for example in the trade products DC 244, 245, 344 and 345 from Dow Corning. Likewise suitable are volatile linear silicone oils, in particular hexamethyldisiloxane (L2), octamethyltrisiloxane (L3), decamethyltetrasiloxane (L4) and any mixtures of two and three of L2, L3 and/or L4, preferably those mixtures as are contained for example in the trade products DC 2-1184, Dow Corning® 200 (about 0.65 cSt) and Dow Corning® 200 (about 1.5 cSt) from Dow Corning. Preferred non-volatile silicone oils are selected from higher-molecular linear dimethylpolysiloxanes, commercially obtainable for example under the name Dow Corning®190, Dow Corning® 200 fluid with kinematic viscosities (about 25° C.) in the range of from about 5-100 cSt, preferably from about 5-50 cSt or also from about 5-10 cSt, and dimethylpolysiloxane with a kinematic viscosity (about 25° C.) of approximately 350 cSt. It may be exceptionally preferred to use mixtures of the aforementioned oils.

It is preferred that the oil comprises an oil of natural origin. It is exceptionally preferred that the oil comprises a plant oil, in particular coconut oil. Coconut oil has a melting point in the range of from about 23 to about 26° C. and belongs to the oils within the scope of this application.

The amount of the at least one oil may be from about 0.5 to about 10% by weight and more preferably from about 1 to about 5% by weight, in each case in relation to the total amount of cosmetic composition.

In a further preferred embodiment the composition also comprises at least one emulsifier.

The at least one emulsifier is preferably non-ionic and an optionally polyethoxylated fatty acid polyol ester of the fatty acid glycol monoester, fatty acid glyceryl monoester, and sorbitol fatty acid ester type. Preferred examples of optionally polyethoxylated fatty acid glycol mono esters are PEG-100 Stearate, PEG-90 Stearate, PEG-50 Stearate, PEG-30 Stearate, PEG-10 Stearate, PEG-7 Stearate, PEG-100) Oleate, PEG-90 Oleate, PEG-50 Stearate, PEG-30 Oleate, PEG-10 Oleate, PEG-7 Oleate, PEG-100 Cocoate, PEG-90 Cocoate, PEG-50 Cocoate, PEG-30 Cocoate, PEG-10 Cocoate, PEG-7 Cocoate.

Preferred examples of optionally polyethoxylated fatty acid glyceryl monoesters are Glyceryl Stearate, PEG-30 Glyceryl Stearate, PEG-20 Glyceryl Stearate, Glycerl Oleate, Glyceryl Cocoate, Glyceryl Palmitate, PEG-18 Glyceryl Oleate, PEG-18 Glyceryl Cocoate, PEG-7 Glyceryl Cocoate, PEG-30 Glyceryl Cocoate, PEG-80 Glyceryl Cocoate.

Preferred examples of optionally polyethoxylated sorbitol fatty acid esters are those known under the INCI names Polysorbate-20 (Sorbitan monolaurate+20 EO). Polysorbate-60 (Sorbitan monostearate+20 EO), Polysorbate-65 (Sorbitan tristearate+20 EO), Polysorbate-80 (Sorbitan monooleate+20 EO), Polysorbate-85 (Sorbitan trioleate+20 EO).

Polyethoxylated fatty acid glyceryl monoesters are preferred emulsifiers from the aforesaid fatty acid polyol esters. A very particularly preferred emulsifier is PEG-7 Glyceryl Cocoate.

The amount of the at least one emulsifier may be from about 1 to about 10% by weight and more preferably from about 2 to about 6% by weight, in each case in relation to the total amount of cosmetic composition.

The cosmetic composition is provided in particular in the form of a pomade and is suitable for temporarily deforming hair, in particular head hair and/or beard hair. The cosmetic composition is provided in particular in the form of a water-based pomade which can be easily removed from the hair treated with it, for example with the use of water and/or a hair cleansing composition, such as a shampoo.

The following example is intended to explain the subject matter of the present disclosure, without limiting it in any way.

### Example

| | % by weight (active |
|---|---|
| Ingredient | substance) |
| 1,2-propanediol | 1 |
| Glycerol | 10 |
| Ceteareth-25 | 30 |
| PEG-7 Glyceryl Cocoate | 5 |
| Citric acid | 0.1 |
| Preservatives | 1 |
| Perfume | 1 |
| Water | to 100 |

The formulation as contemplated herein was applied by hand on test subjects in order to fix the shape of the hairstyle. The hair was provided with a natural shine and a strong, but flexible hairstyle hold. The hair of the test subjects was at all times soft, silky and easy to comb. In addition, the test subjects reported a positive feel of the hair.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for temporarily deforming hair, comprising—in each case in relation to the total weight of the composition—
a) from about 20 to about 60% by weight water,
b) from about 2 to about 20% by weight of at least one poly valent alcohol, and
c) from about 20 to about 65% by weight of at least one alkoxylated fatty alcohol.

2. The cosmetic composition according to claim 1, wherein the at least one alkoxylated fatty alcohol is selected from the group consisting of Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Laureth-1, Laureth-2, Laureth-3, Laureth-4, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-23, Laureth-25, Laureth-30, Laureth-40, Deceth-3, Deceth-5, Oleth-5, Oleth-30, Steareth-2, Steareth-6, Steareth-7, Steareth-10, Steareth-11 Steareth-13, Steareth-14, Steareth-15, Steareth-20, Steareth-21, Steareth-25, Steareth-27, Steareth-30, Steareth-40, Steareth-50, Steareth-100 and mixtures thereof.

3. The cosmetic composition according to claim 1, wherein the at least one alkoxylated fatty alcohol comprises Ceteareth-25.

4. The cosmetic composition according to claim 1, wherein
the at least one alkoxylated fatty alcohol is Ceteareth-25.

5. The cosmetic composition according to claim 1, wherein the at least one polyvalent alcohol is selected from the group consisting of 1,2-propanediol, 1,3-propanediol, 2-methyl-1,3-propanediol, glycerol, 1,2-butylene glycol, 1,3-butylene glycol, 1,4-butylene glycol, 1,2-pentanediol, 1,5-pentanediol, 1,2-hexanediol, 1,6-hexanediol, 2-methyl-2,4-pentanediol, 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomer mixtures of eis- and trans-1,4-dimethylolcyclohexane, PEG-3, PEG-4, PEG-6, PEG-7, PEG-8, PEG-9, PEG-10, PEG-12, PEG-14, PEG-16, PEG-18 and PEG-20 and mixtures thereof.

6. The cosmetic composition according to claim 5, wherein the at least one polyvalent alcohol comprises glycerol or a mixture of 1,2-propanediol and glycerol.

7. The cosmetic composition according to claim 1, wherein the composition also comprises at least one oil.

8. The cosmetic composition according to claim 1, wherein the composition also comprises at least one emulsifier.

9. Use of a cosmetic composition according to claim 1 for temporarily deforming keratinous fibres.

10. A method for temporarily deforming hair comprising applying a cosmetic composition according to claim 1 to the hair.
